(19) United States
(12) Patent Application Publication
(10) Pub. No.: US 2016/0051260 A1
(43) Pub. Date: Feb. 25, 2016

(54) SYSTEM FOR JOINING TWO BLOOD VESSELS

(71) Applicant: Xavier RENARD, (US)

(72) Inventors: Xavier RENARD, Vauhallan (FR); Bruno MASONI, Ozieres (FR)

(21) Appl. No.: 14/781,503

(22) PCT Filed: Mar. 24, 2014
(86) PCT No.: PCT/FR2014/000060

§ 371 Date: Sep. 30, 2015

(30) Foreign Application Priority Data
Apr. 2, 2013 (FR) 13/00751

(51) Int. Cl. A61B 17/11; A61B 17/122

(52) U.S. Cl. CPC A61B 17/11; A61B 17/1227; A61B 2017/00477

(57) ABSTRACT

A system for bringing two blood vessels into abutment includes two clamps Pc and a rod 260 connecting the two clamps together by being engaged in two through holes 250 made respectively in the two clamps, a portion of the rod lying between the two clamps possessing two moments of inertia Mi1, Mi2 of different values along two mutually orthogonal directions, one of which is perpendicular to the first plane P1, the smaller value moment of inertia Mi1 being that defined substantially along the direction that is perpendicular to the first plane P1. The system is applicable to bringing two blood vessels into abutment, in particular for the purpose of suturing them.

The present invention relates to systems for bringing two blood vessels into abutment by means of vascular clamps.

Such clamps are commonly used in surgery, in particular of the hand or of the foot, whenever it is necessary to manipulate, hold, bring into abutment, etc., small blood vessels during a surgical operation, e.g. for suturing purposes.

Such clamps are already known, e.g. as described and shown in EP 0 105 414, and also in U.S. Pat. No. 5,103,838 and WO 2004/073487, for example.

Such a clamp essentially comprises two substantially identical tabs, means for mounting the tabs to pivot relative to each other about a pivot axis situated substantially in the proximity of the middle portions of the two tabs, thereby defining first and second portions on each tab that are situated on either side of a plane containing the pivot axis and substantially perpendicular to the general plane of at least one of the tabs, and means for applying two respective opposing resilient forces on the first tab portions so that the two free ends of the first tab portions tend to move apart from each other and, correspondingly, the free ends of the two second tab portions tend to come into contact with each other.

An embodiment as described above gives the expected results with relative satisfaction. Nevertheless, it presents the major drawback of giving the system a cost price that is relatively high, given that this type of vascular clamp is a device for single use and that it sometimes impedes the practitioner during the suturing operation.

Thus, an object of the present invention is to provide a system for bringing two blood vessels into abutment that tends to mitigate at least in part the above-mentioned drawbacks of the prior art as described above, and that can also satisfy certain desires of surgeons, e.g. giving them the possibility of selecting or modifying the pressure exerted by the clamp on the vessel that it is to hold, enabling surgeons to apply a pressure force that can be varied in a direction defined by the longitudinal axis of bundles of blood vessels in order to enable them to be sutured.

However, and above all, another object of the present invention is to provide a system that makes the operating work of practitioners easier when suturing together two blood vessels.

More precisely, the present invention provides a system for bringing two blood vessels into abutment, the system being characterized in that it comprises least two clamps, each clamp comprising: two substantially identical tabs, means for pivotally mounting said two tabs relative to each other about a pivot axis, said pivot axis being situated substantially in the proximity of respective middle portions of the two tabs, thereby defining on each of the tabs first and second portions situated on either side of a first plane containing said pivot axis and substantially perpendicular to the general plane of at least one of the two tabs; means for applying two respective opposing resilient forces on the two first tab portions so that the two free ends of these two tab portions tend to move apart from each other and, correspondingly, the free ends of the two second tab portions tend to come into contact with each other; and a through hole made in one of the two tabs along an axis substantially parallel to said pivot axis, said through hole being cylindrical and of polygonal cross-section; said system further including a rod of longitudinal axis and having a cross-section that is complementary to the cross-section of the through hole, said rod being engaged in the two respective through holes of the two clamps, the system being characterized by the fact that a portion of said rod extending between the two clamps possesses two moments of inertia of different values in two mutually orthogonal directions, one of which is perpendicular to the first plane, the smaller value moment of inertia being the moment of inertia defined along the direction that is perpendicular to said first plane.

Other characteristics and advantages of the present invention appear from the following description by way of non-limiting illustration with reference to the accompanying drawings, in which:

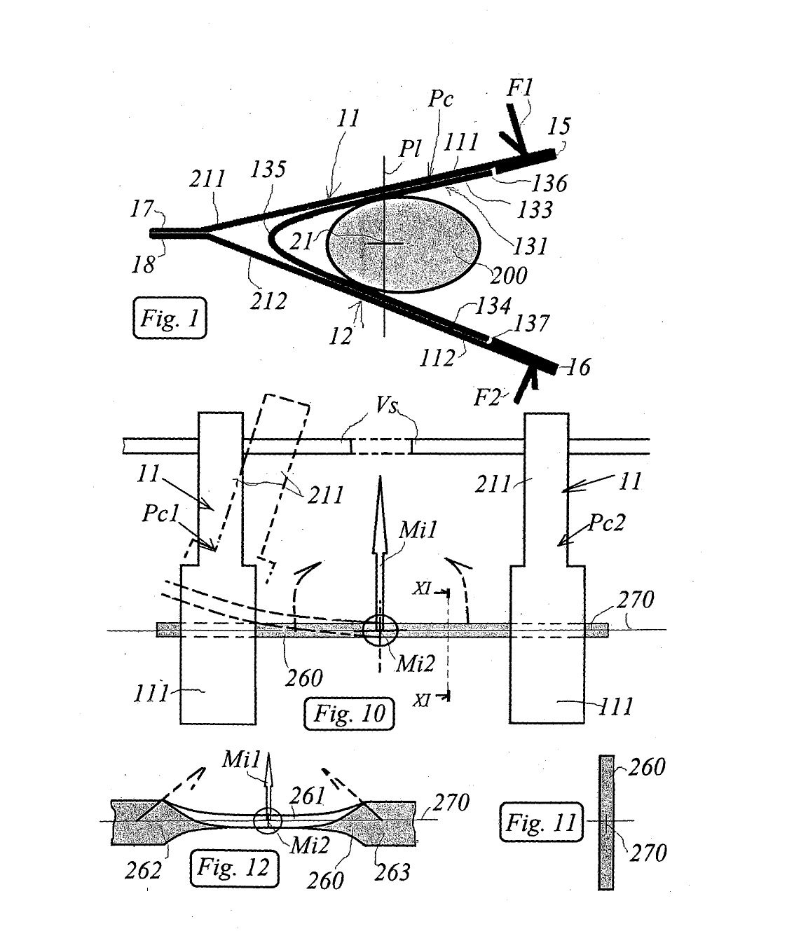

FIG. 1 is a diagram showing the principle of a vascular clamp used for constituting the system of the invention for bringing at least two blood vessels into abutment;

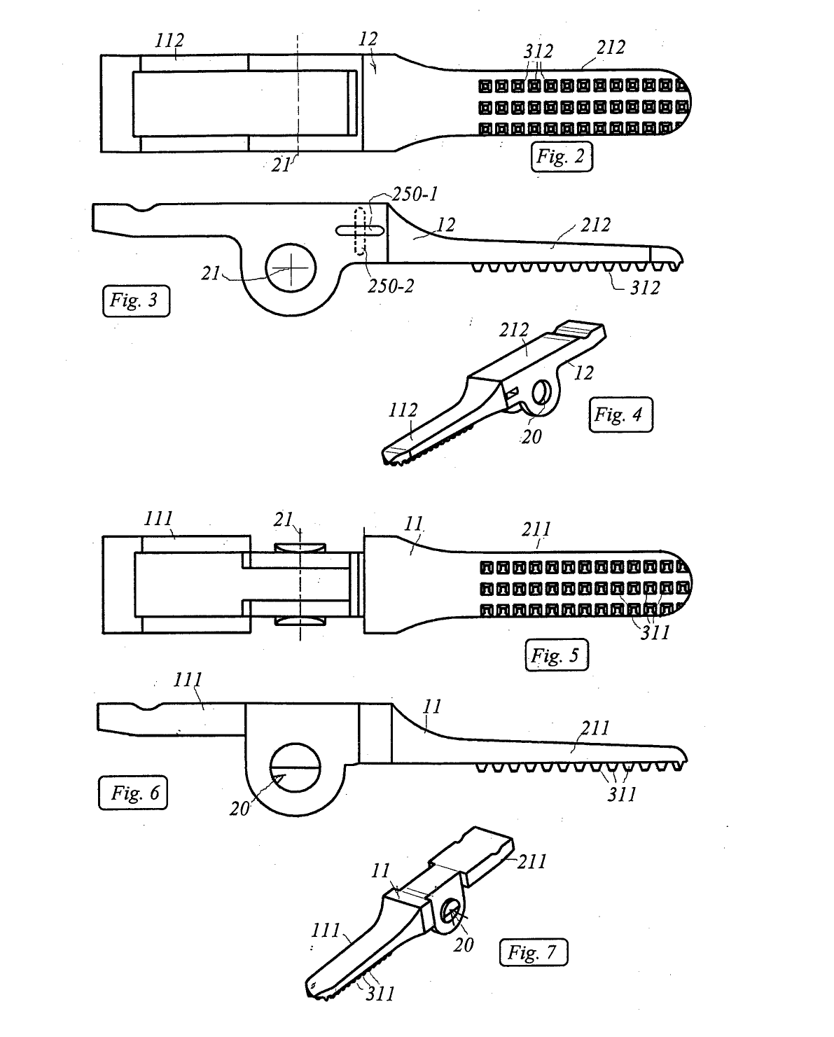

FIGS. 2, 3, and 4 show one of the component elements of the FIG. 1 vascular clamp respectively in views from below, from the side, and in perspective;

FIGS. 5, 6, and 7 show another of the component elements of the FIG. 1 vascular clamp, respectively in views from below, from the side, and in perspective;

FIG. 8 is an exploded perspective view of an embodiment of the vascular clamp of FIGS. 1 to 7;

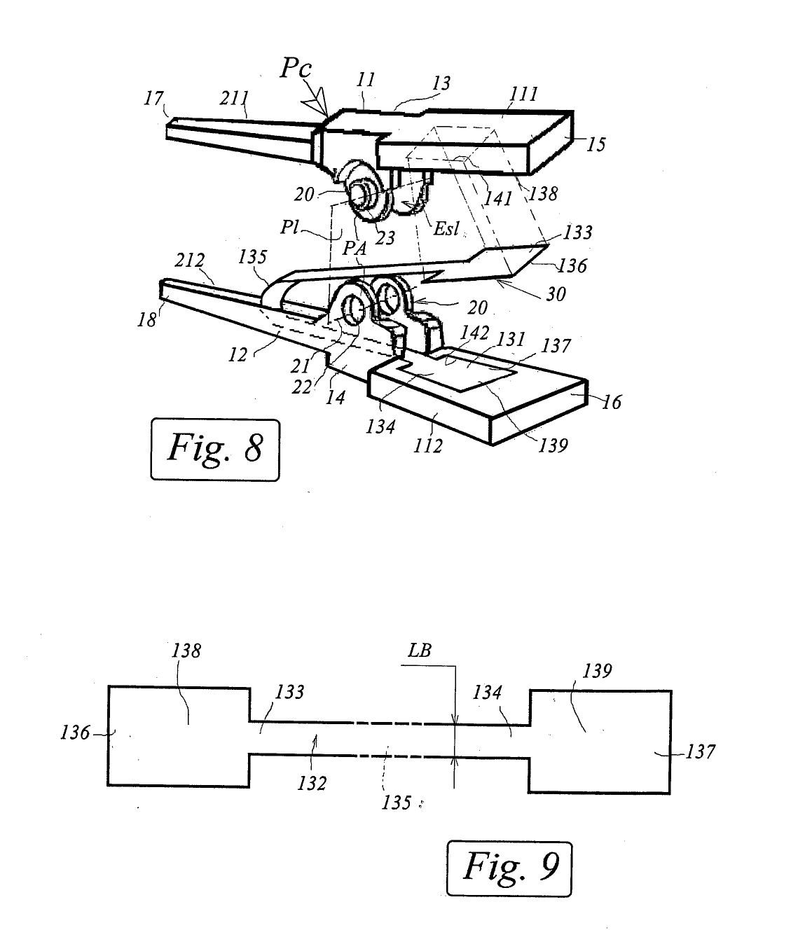

FIG. 9 is a plan view of an element suitable for making one of the component elements of the FIG. 8 vascular clamp; and

FIGS. 10, 11, and 12, taken in combination, show two embodiments of a system of the invention for bringing two blood vessels into abutment, the system comprising two vascular clamps of FIGS. 1 to 8 connected together by a rod, FIG. 10 being a plan view, FIG. 11 being a view in the section referenced XI-XI in FIG. 10, and FIG. 12 being a plan view showing one possible embodiment of one of the elements shown in FIG. 10.

It is specified initially that in the present description if the adverb “substantially” is associated with a qualification of any given means, the qualification should be understood either strictly or else approximately.

Furthermore, the scale with which certain elements are shown in the figures is not uniform. For certain details of those elements, the scale used, e.g. in FIG. 10, is not the same along two non-coinciding directions. This type of representation has been adopted to show clearly, by exaggeration, the structural characteristics of the various elements.

With reference to the accompanying figures, the present invention relates to a system for bringing at least two blood vessels Vs into abutment, the system comprising two vascular clamps Pc, e.g. for the purpose of suturing the two blood vessels.

Each vascular clamp Pc comprises two substantially identical tabs 11 and 12 together with means 20 for mounting these two tabs to pivot relative to each other about a pivot axis 21 situated substantially in the proximity of the respective middle portions 13 and 14 of the two tabs.

These pivot means 20 may for example be of the same type as the means described and shown in the prior document mentioned in the introduction of the present description.

As shown in FIGS. 2 to 8, these pivot means 20 are advantageously constituted by at least one pivot system PA comprising a bearing 22 and a shaft 23 complementary to the bearing, the bearing being constituted by a cylindrical ring substantially in the form of a body of revolution around the pivot axis 21 so that the shaft 23 can be clipped in the ring. However, in even more preferred manner, these means 20 comprise two substantially identical pivot systems PA that are spaced apart from each other so as to leave an empty space Es1 between them.

As a result, these pivot means 20 define, on each of the two tabs 11 and 12, both a first portion 111, 112 and a second portion 211, 212, which portions are situated on either side of a plane P1 containing the pivot axis 21 and substantially perpendicular to the general plane of at least one of the two tabs.

The vascular clamp Pc also includes means 30 for applying two opposing resilient forces respectively against the two first portions of the tabs 111 and 112 so that the two free ends 15 and 16 of these first tab portions tend to move apart from each other, and correspondingly the free ends 17, 18 of the two second tab portions 211 and 212 tend to come into contact with each other.

These means 30 for applying two opposing resilient forces on the two first tab portions 111, 112 are constituted by a substantially U-shaped part 131 formed in a plate 132 of an elastic material that is folded so as to have two branches 133, 134, with one end of one of the two branches being connected to one end of the other branch by a half-loop 135 that is open over at least ninety degrees of angle, the U-shaped part being engaged between the two tabs 11 and 12.

In order to obtain both static and dynamic equilibrium for the clamp, the open half-loop 135 is preferably positioned substantially in the proximity of the pivot axis 21.

In the meaning of the present description, the term “plate” should be understood to be an oblong element that is relatively flat and of width that is clearly greater than its thickness.

The vascular clamp Pc also includes means for bringing the two free ends 136, 137 of the two branches 133, 134 of the U-shaped part into abutment respectively against facing faces of the two first tab portions 111, 112 so that the U-shaped part does not tend, on relaxing naturally, to slide out from the V-shaped space defined between the two tabs 11 and 12, see FIG. 1.

These means for bringing the two free ends 136, 137 of the two branches 133, 134 into abutment respectively against the facing faces of the two first tab portions 111, 112 are essentially constituted by two recessed housings 141, 142 formed in the facing faces of these first tab portions 111, 112 that receive the respective free ends 136, 137 of the two branches 133, 134 of the U-shaped part.

In a particularly advantageous embodiment, these free ends 136, 137 of the two branches 133, 134 of the U-shaped part are shaped as enlarged portions 138, 139 that are wider than the reminder of the plate 132, and the housings are of shape complementary to these enlarged portions so that they become respectively engaged in congruent manner in the housings 141, 142.

In the above-described embodiment, the two branches 133, 134 of the U-shaped part 131 take up positions in the above-defined space Es1 and, according to a characteristic of the invention, at least one of these two branches 133, 134, and advantageously both of them, present(s) a width LB that is substantially equal to the width of the empty space Es1.

Thus, when the two “bearing 22 and shaft 23” assemblies of the two respective pivot systems PA are assembled together, the branches prevent the bearings 22 and the shafts 23 from separating, because of these branches having the above-defined width.

In a preferred embodiment, see FIGS. 3 to 7, each of the faces of the second tab portions 211, 212 that face each other includes a plurality of projecting studs 311, 312, these studs being arranged on these faces in such a manner that the studs situated on the face of one of the second tab portions 211, 212 are arranged in staggered configuration relative to the studs situated on the face of the other second tab portion 211, 212 when these second tab portions come substantially into contact with each other under the action of the opposing resilient forces applied to the first tab portions 111, 112.

In another possible characteristic of the invention, the system may also include at least one elastomer block 200 that is elastically deformable in compression, this block having dimensions that enable it to be inserted by force, by being deformed, between the two first tab portions 111, 112.

In addition, the block 200 is advantageously situated between the two branches 133, 134 of the U-shaped part 131, and even in the proximity of, but not in contact with, the open half-loop 135.

In order to make it easier to use, the block 200 preferably presents a shape selected from the following two shapes: a cylinder of substantially elliptical cross-section, see FIG. 1, with focal axes substantially parallel to the pivot axis of rotation 21; and a truncated pyramid inscribed in a dihedron having its ridge substantially parallel to the pivot axis 21 and arranged in such a manner that the two faces defining the dihedron come into contact respectively with the facing faces of the two branches 133, 134 of the U-shaped part 131.

This embodiment is advantageous since it enables the practitioner to adjust freely the value of the pressure that can be obtained at the free ends 17, 18 of the two second tab portions 211, 212 in order to obtain the desired intensity for the clamping force that is applied to the blood vessels Vs, either by not using a block 200 as described, or else by inserting such a block between the two branches 133, 134 of the U-shaped part 131 if the practitioner seeks to increase the pressure exerted on the blood vessels. Advantageously, this embodiment of the system of the invention thus makes the practitioner free to select, e.g. by means of predetermined charts, one block 200 from a set of blocks of different shapes that are made available in order to obtain desired pressure on the blood vessels that are to be brought into abutment.

Advantageously, to make the system of the invention easier for the practitioner to use during surgery, one of the two tabs 11, 12 of each clamp Pc1, Pc2 includes a cylindrical through hole 250 of polygonal cross-section extending along an axis substantially parallel to the pivot axis 21, and the system also includes a rod 260 of longitudinal axis 270 and of cross-section complementary to that of the through hole, the rod being engaged in the two through holes 250 in respective ones of two clamps with a degree of friction that does not enable the clamps to slide easily along the rod.

According to a characteristic of the invention, at least a portion of the rod 260 extending between the two clamps Pc1, Pc2 possesses two second moments of area (or “moments of inertia”) Mi1 and Mi2 of different values in two mutually orthogonal directions, one of which is perpendicular to the first plane P1, the moment of inertia Mi1 of smaller value being that which is defined substantially along the direction that is perpendicular to the first plane P1, and the other moment of inertia Mi2 thus being that which is defined along a direction substantially parallel to the plane P1.

In a possible embodiment, see FIGS. 10 and 11, the rod 260 is constituted by a plane plate defined over its entire length in a single plane and having thickness that is less than its width, see FIG. 11, and the cross-section of the through hole 250-1 is substantially rectangular, this through hole presenting a depth defined in a direction parallel to the pivot axis 21, and a width and a length defined respectively in two directions that are perpendicular to the pivot axis 21. In this embodiment of the rod 260, the length of the through hole 250-1 is substantially parallel to the first plane P1, as shown in dashed lines in FIG. 3.

In another possible embodiment, see FIGS. 10 and 12, the rod 260 is constituted by a plate, see FIG. 12, having a middle first portion 261 defined in a plane and situated between two second portions 262, 263 defined in a plane substantially perpendicular to the plane of the first portion. The cross-section of the through hole 250-2, like the cross-section of the through hole 250-1, is substantially rectangular, however its length is substantially perpendicular to the first plane P1, as shown by a continuous line in FIG. 3, the two second portions 262, 263 of the rod then being engaged respectively in the two respective through holes 250-2 of the two clamps.

According to a characteristic of the invention, in order to achieve the object of the invention, the rod 260 is made of a material that presents negligible elastic deformation when subjected to twisting forces, so that plastic deformation predominates, e.g. being made of stainless steel or the like.

In addition to the explanations given above, the system of the invention for bringing at least two blood vessels Vs into abutment and including at least two clamps Pc1, Pc2 is used as follows:

Initially, while at rest, the two second tab portions 211, 212 of the clamps Pc are in contact with each other via their free ends 17, 18, while the free ends 15, 16 of the two first tab portions 111, 112 are maximally spaced apart from each other.

In order to clamp onto one of the two blood vessels, the practitioner presses in opposite directions F1, F2 on the free ends 15, 16 of the first tab portions in order to move them towards each other against the resilient force exerted by the U-shaped part and the elastomer block 200, if present, which becomes even more compressed. Consequently, the free ends 17, 18 of the second tab portions move apart from each other and the practitioner can place them on either side of the blood vessel. When the practitioner releases the ends 15, 16, the two ends 17, 18 move towards each other and clamp the blood vessel.

In order to bring two blood vessels Vs into abutment, it is necessary to use at least two vascular clamps in parallel, by coupling them together on a rod 260, as described above with reference to FIG. 10.

Depending on circumstances and on its structure as shown in FIG. 10, the system is used as follows:

The two clamps Pc1, Pc2 are positioned on the rod 260 as described above and at a distance apart from each other that is greater than when using prior art solutions, and then they are clamped on respective ones of the two blood vessels Vs, see FIG. 10, e.g. having sectioned ends for suturing together, so that their separate ends are close to each other.

The rod 260 is then bent about an axis parallel to the plane P1, either manually or by using an appropriate ancillary instrument, and this is relatively easy to do because of the structure of the rod of the invention, in particular because its smaller moment of inertia Mi1 is defined in a direction substantially perpendicular to the plane P1.

This bending continues until the two sectioned ends of the two blood vessels come into contact with each other with a certain amount of pressure.

In the above-described process, the free ends of the second tab portions of the two clamps are closer together than are the free ends of the first tab portions.

Thus, the two tabs are in an oblique position relative to each other, as shown in dashed lines for the clamp Pc1 and in continuous lines for the clamp Pc2 in FIG. 10. This arrangement makes it possible to define an empty space between the clamps that is much greater than with prior art solutions, even though the free ends of the first tab portions can be brought as close together as with those prior solutions, thus giving the practitioner more access to the two ends of the blood vessels for suturing together than is available when the two clamps are parallel to each other. The practitioner can then suture the two vessel ends together with greater ease and safety.

It is nevertheless possible to use the rod 260 by bending its middle portion 261 through about three hundred and sixty degrees, so that it forms a closed loop with the two second tab portions 262, 263 outside the loop forming between them a right angle, for example. The two clamps Pc are then positioned on these two portions. This configuration is very advantageous, e.g. for making lateral terminal vascular connections, in particular vertically, e.g. so as to enable two practitioners to operate together.

The above description shows clearly the advantages presented by the system of the invention for bringing two blood vessels into abutment in comparison with prior arts systems, and shows that the objects of the present invention are achieved.

1. A system for bringing two blood vessels (Vs) into abutment, the system comprising at least two clamps (Pc), each clamp comprising: two substantially identical tabs (11, 12), means (20) for pivotally mounting said two tabs (11, 12) relative to each other about a pivot axis (21), said pivot axis (21) being situated substantially in the proximity of respective middle portions (13, 14) of the two tabs, thereby defining on each of the tabs first and second portions (111, 112; 211, 212) situated on either side of a first plane (P1) containing said pivot axis (21) and substantially perpendicular to the general plane of at least one of the two tabs; means (30) for applying two respective opposing resilient forces on the two first tab portions (111, 112) so that the two free ends (15, 16) of these two tab portions (111, 112) tend to move apart from each other and, correspondingly, the free ends (17, 18) of the two second tab portions (211, 212) tend to come into contact with each other; and a through hole (260) made in one of the two tabs along an axis substantially parallel to said pivot axis (21), said through hole being cylindrical and of polygonal cross-section; said system further including a rod (260) of longitudinal axis (270) and having a cross-section that is complementary to the cross-section of the through hole, said rod being engaged in the two respective through holes (250) of the two clamps, wherein a portion of said rod extending between the two clamps possesses two moments of inertia of different values in two mutually orthogonal directions, one of which is perpendicular to the first plane (P1), the smaller value moment of inertia being the moment of inertia defined along the direction that is perpendicular to said first plane (P1).

2. A system according to claim 1, wherein said rod (260) is constituted by a plane plate defined over its entire length in a single plane (FIG. 10) and having thickness that is less than its width, and the cross-section of the through hole (250-1) is substantially rectangular, said through hole presenting a depth defined in a direction parallel to said pivot axis (21), and a width and a length defined respectively in two directions that are perpendicular to said pivot axis (21), its length being substantially parallel to the first plane (P1).

3. A system according to claim 1, wherein said rod (260) is constituted by a plate having a middle first portion (261) defined in a plane and situated between two second portions (262, 263) defined in a plane substantially perpendicular to the plane of the first portion, and the cross-section of the through hole (250-2) is substantially rectangular, said through hole presenting a depth defined in a direction parallel to said pivot axis (21), and a width and a length defined respectively along two directions perpendicular to said pivot axis (21), its length being substantially perpendicular to the first plane (P1), the two second portions (262, 263) of the rod being engaged respectively in the two respective through holes (250-2) of the two clamps.

4. A system according to claim 1, wherein said rod (260) is made of a material that presents negligible elastic deformation when subjected to twisting forces, so that plastic deformation predominates.

5. A system according to claim 1, wherein said means (30) for applying two opposing resilient forces respectively against the two first portions of the tabs (111, 112), are constituted by a substantially U-shaped part (131) formed in a plate (132) made of an elastic material that is folded so as to have two branches (133, 134), one end of one of the two branches being connected to one end of the other branch by a half-loop (135) that is open over at least one hundred and eighty degrees of angle, said U-shaped part being engaged between the two tabs (11, 12), and said clamp also includes means for connecting together in abutment the two free ends (136, 137) of the two respective branches (133, 134) respectively with the facing faces of the two first tab portions (111, 112).

6. A system according to claim 5, wherein the means for bringing the two free ends (136, 137) of the two branches (133, 134) into abutment respectively against the facing faces of the two tab portions (111, 112) are constituted by recessed housings (141, 142) made in the facing faces of these first tab portions (111, 112) that receive the respective free ends (136, 137) of the two branches (133, 134) of said U-shaped part.

7. A system according to claim 1, wherein each of the faces of the second tab portions (211, 212) that face each other includes a plurality of projecting studs (311, 312), said studs being arranged on said faces in such a manner that the studs situated on the faces of one of the second tab portions (211, 212) are arranged in staggered configuration relative to the studs situated on the face of the other second tab portion (212, 211) when these second tab portions come substantially into contact with each other under the action of the opposing resilient forces applied to the first tab portions (111, 112).

8. A system according to claim 5, wherein said means (20) for mounting said branches (11, 12) to pivot relative to each other about said pivot axis (21) are constituted by at least one pivot system (PA) comprising a bearing (22) and a shaft (23) complementary to said bearing, said bearing being constituted by a cylindrical ring substantially in the form of a body of revolution around said pivot axis (21) so that said shaft (23) can be clipped in said ring.

9. A system according to claim 8, further comprising two substantially identical pivot systems (PA), said two pivot systems being spaced apart from each other so as to leave an empty space (Es1) between them, in which said two branches (133, 134) take up position.

10. A system according to claim 9, wherein at least one of the two branches (133, 134) of said substantially U-shaped part (131) has a width (LB) that is substantially equal to the width of said empty space (Es1).

11. A system according to claim 3, wherein said middle portion (261) of the rod (260) is bent so as to form a closed loop and that said two second portions (262, 263) are situated outside said loop.

12. A system according to claim 2, wherein said means (30) for applying two opposing resilient forces respectively against the two first portions of the tabs (111, 112), are constituted by a substantially U-shaped part (131) formed in a plate (132) made of an elastic material that is folded so as to have two branches (133, 134), one end of one of the two branches being connected to one end of the other branch by a half-loop (135) that is open over at least one hundred and eighty degrees of angle, said U-shaped part being engaged between the two tabs (11, 12), and said clamp also includes means for connecting together in abutment the two free ends (136, 137) of the two respective branches (133, 134) respectively with the facing faces of the two first tab portions (111, 112).

13. A system according to claim 12, wherein the means for bringing the two free ends (136, 137) of the two branches (133, 134) into abutment respectively against the facing faces of the two tab portions (111, 112) are constituted by recessed housings (141, 142) made in the facing faces of these first tab portions (111, 112) that receive the respective free ends (136, 137) of the two branches (133, 134) of said U-shaped part.

14. A system according to claim 2, wherein each of the faces of the second tab portions (211, 212) that face each other includes a plurality of projecting studs (311, 312), said studs being arranged on said faces in such a manner that the studs situated on the faces of one of the second tab portions (211, 212) are arranged in staggered configuration relative to the studs situated on the face of the other second tab portion (212, 211) when these second tab portions come substantially into contact with each other under the action of the opposing resilient forces applied to the first tab portions (111, 112).

15. A system according to claim 1, wherein said means (20) for mounting said branches (11, 12) to pivot relative to each other about said pivot axis (21) are constituted by at least one pivot system (PA) comprising a bearing (22) and a shaft (23) complementary to said bearing, said bearing being constituted by a cylindrical ring substantially in the form of a body of revolution around said pivot axis (21) so that said shaft (23) can be clipped in said ring.
